(19) United States
(12) Patent Application Publication
(10) Pub. No.: US 2015/0080861 A1
(43) Pub. Date: Mar. 19, 2015

(54) SPONGE SHEATH FOR LAPAROSCOPIC ASPIRATOR

(71) Applicant: M. Tahir OZER, (US)

(72) Inventor: M. Tahir Özer, Ankara (TR)

(21) Appl. No.: 14/382,873

(22) PCT Filed: Mar. 7, 2013
(86) PCT No.: PCT/TR2013/000077

§ 371 Date: Sep. 4, 2014

(30) Foreign Application Priority Data
Mar. 8, 2012 (TR) 2012/02653

(51) Int. Cl. A61M 1/00; A61B 17/00; A61F 13/38

(52) U.S. Cl. CPC A61M 1/0039; A61F 13/38; A61M 1/008; A61B 17/00234; A61M 2205/0238; A61B 2017/00477
USPC 604/540

(57) ABSTRACT

The present invention relates to the aspirators, one of the hand tools used in laparoscopic surgery. In the present invention, a sponge sheath that can be fitted on the known aspirator tip structure in order to prevent blockage that may occur in the aspirators by adherence of the bowel and omentum during the aspiration of the interior of the abdomen.

The present invention relates to the aspirators, one of the hand tools used in laparoscopic surgery. In the present invention, a sponge sheath that can be fitted on the known aspirator tips in order to prevent blockage that may occur in the aspirators during the aspiration of the interior of the abdomen is described.

Washing and aspiration of the interior of the abdomen is required in many stages of the surgeries in all the surgeries of the surgical branches. With the washing and aspiration process, the infected and necrotic material inside the abdomen is removed therefrom or diluted. In said aspiration process, laparoscopic aspirator devices are used if the surgery is laparoscopic. The intra-abdominal organs, especially the bowel and omentum adhere to the tip of the aspirator during the aspiration process and blocks said process. In this case, the surgeon tries to protect the tip of the aspirator from the bowel and omentum by using their hand and auxiliary tools. In the laparoscopic surgery applications where the surgeon cannot introduce their hand into the abdomen and realizes the avoidance maneuvers in a more difficult manner, blockage of the aspirator tip by the intra-abdominal organs, especially the bowel and omentum, creates problems from time to time. In the existing laparoscopic aspirators, no special system preventing the blockage of the aspiration is provided. Thus, the surgeon tries to carry out the aspiration processes by keeping the bowel and omentum away from the operation area without approximating the aspirator tip to the bowel and omentum and by means of gravity effect through positioning the operation table when required. However, in this case, the fluids desired to be aspirated are collected in the deepest part of the abdomen along with the bowel and omentum, thus, the fluids desired to be kept away from the abdomen cannot be completely removed. The inability to remove the fluids from the abdomen increases the risk of infection, intra-abdominal abscess and intra-abdominal adhesion and ileus risk associated therewith since said fluids desired to be removed from the abdomen contain infected and necrotic materials. These situations may lead to reoperations. Drainage tubes are placed into the abdomen at the end of the operation for removing the remaining infected and necrotic material.

The drainage tubes are drawn when draining stops. The drainage tube stays inside the abdomen as long as the fluid and infected material is present therein and the long duration of the drainage tube remaining inside the abdomen can lead to ascending infection inside the abdomen (inwards from outside through the drainage tube). As a result, the risk increases proportionally with amount of the infected and necrotic material remaining in the abdomen after the operation.

Due to the aforementioned difficulties during the operation, the prolonging of the aspiration process also prolongs the operation duration and causes the patient to be under anesthesia for longer period of time.

The object of the present invention is to allow sustaining the aspiration without any interruption and blockage between the intestinal loops and even on the omentum thanks to fitting the developed sponge sheath on the standard aspirator tip. Thus, the unwanted fluid material can be completely taken out of the abdomen and development of intra-abdominal abscess and infection as well as ileus development risk associated with the adhesions can be reduced. Realization of the aspiration process smoothly and quickly will shorten the duration of surgery.

In the patent application FR 2 170 858 (A1), relates to improvements in a medical drain is disclosed. The drain comprises a generally tubular sponge trapped between a perforated collecting tube and also a perforated sleeve, the assembly being made of relatively soft material like sponge. But the device mentioned at FR 2 170 858 (A1) have a tubular which is getting narrower at the suction end of the drain.

In the patent application WO 2011/038949 (A1), relates to a vacuum sponge unit for use in human or animal bodies, comprising a fluid collecting element, in particular a sponge unit, and a fluid communication element, in particular a drainage hose, which is at least partially arranged in the fluid collecting element and is connected to the fluid collecting element in a fluid conducting manner. In order to improve known devices, according to the invention the vacuum sponge unit is further developed, wherein the fluid collecting element comprises a channel for guiding a guide element, in particular a guide wire, and/or a supply element, in particular a probe unit and/or an endoscope, through the fluid collecting element.

In the patent application US 2005/171467(A1), relates to a surgical device providing multiple functions including aspiration, irrigation, traction, filtration, dissection and compression of tissue, the surgical device comprising an elongate shaft having a proximal end and a distal end, a mobilization tip operatively attached at the distal end of the elongate shaft for manipulating tissue, and a valve assembly operatively connected to the proximal end of the elongate shaft for selectively delivering and removing an irrigation fluid to and from a surgical site through the elongate shaft. The surgical device is operable with one hand. The surgical device is dimensioned according to its use in either open or minimally invasive surgery. The mobilization tip comprises a traction-enhancing material formed of reticulated foam or from a woven or braided fabric. The mobilization tip further includes a porous filter for preventing biological matters from being inadvertently drawn into the aspiration holes or windows at the distal end of the elongate shaft.

In the patent application EP 2156853(A1), relates to a medical product used for treatment of sinusitis and comprising an absorption body (12) for bodily fluids.

Such designs will increase the risk of unintentional blocking or interruption of the aspiration process due to intra-abdominal organs adhering to and blocking suction tip. This because of the tubular or conic design design of the suction tip.

### DESCRIPTION OF THE FIGURES

FIG. 1. Longitudinal cross sectional view of the sponge sheath for laparoscopic aspirator.

FIG. 2. Front view of the front tip of the sponge sheath for laparoscopic aspirator.

FIG. 3. Front view of the rear tip of the sponge sheath for laparoscopic aspirator.

FIG. 4. View of the sponge sheath for laparoscopic aspirator attached to the conventional aspirator.

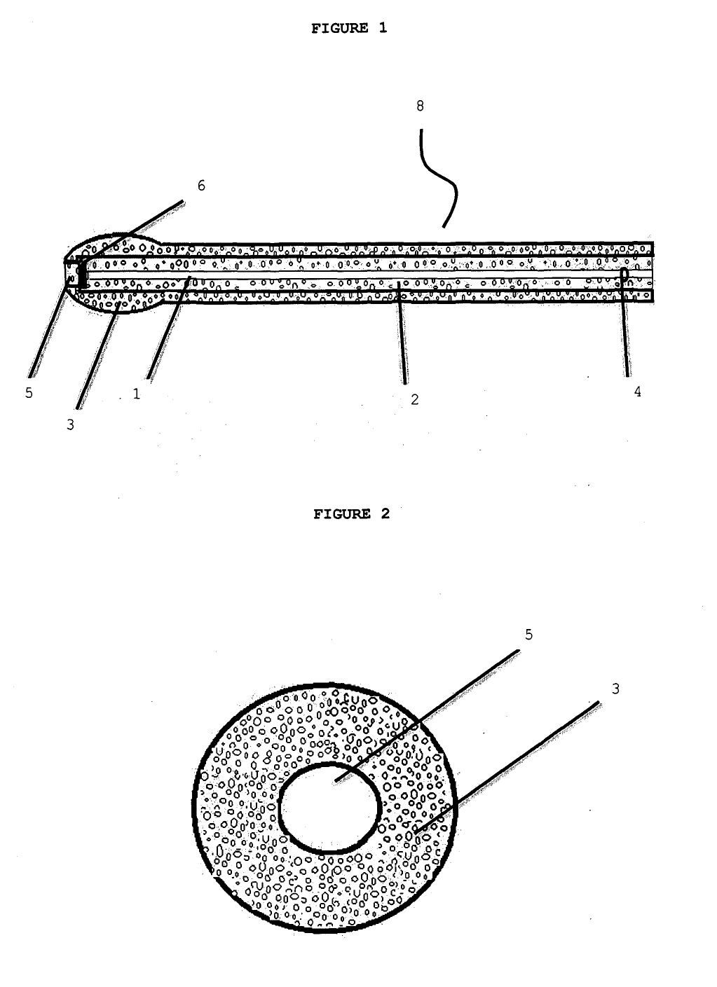

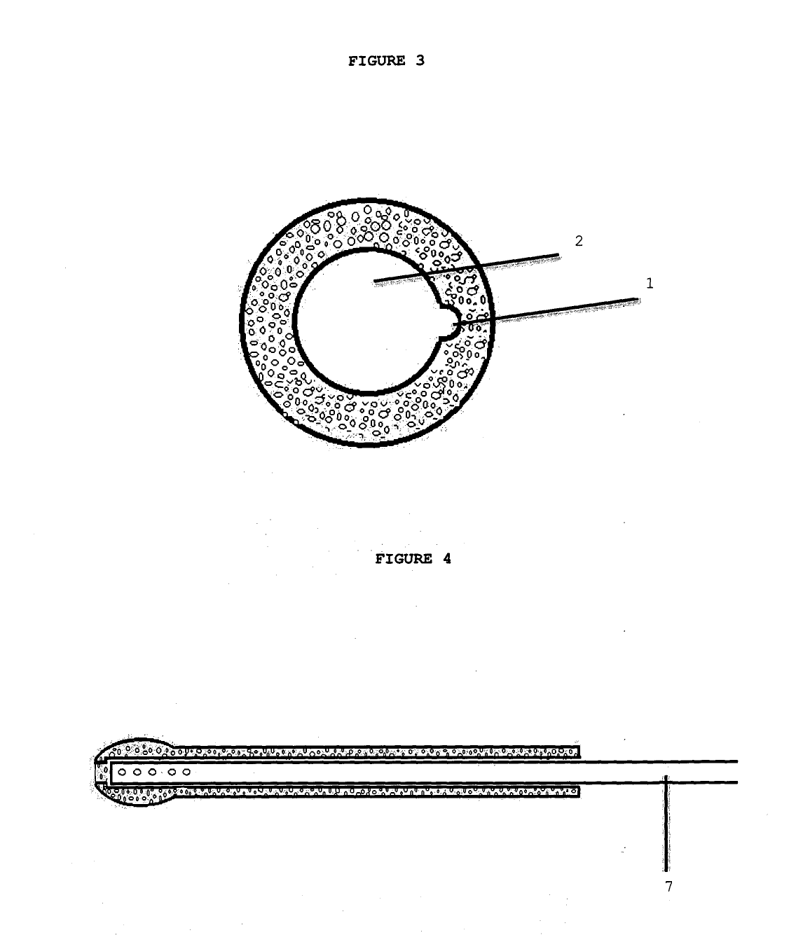

### PART NUMBERS

1. Air channel
2. Aspirator channel
3. Sponge sheath tip
4. Air channel connection outlet
5. Main aspiration channel
6. Support ring
7. Aspirator tip
8. Upper body
9. Suction end

The sponge sheath for laparoscopic aspirator according to the present invention is a specially shaped apparatus being preferably made of large pore sponge and attached to the conventional aspirator tip (7).

The sponge sheath according to the present invention that can be used for laparoscopic, aspirators is provided with the following: an upper body (8) extending across the whole body except for sponge sheath tip (3); a sponge sheath tip (3) provided in one end portion of said upper body (8) and designed in the form of an ellipse having open ends; a main aspiration channel (5) provided in suction end (9) of the sponge sheath tip (3) and designed as a cylindrical channel having a diameter smaller than the diameter of the aspirator channel (2); a support ring (6) positioned between the aspirator channel (2) and the main aspiration channel (5); an air channel (1) positioned in the inner part of the aspirator channel in the form of a smooth semi-cylindrical channel; and an air channel connection outlet (4) connecting the air channel (1) and the aspirator channel (2) to the external environment.

The above-described air channel connection outlet (4) connects the aspirator channel (2) as well as the air channel (1) to the intra-abdominal medium inflated with carbon dioxide. The air channel connection outlet (4) is positioned so as to be close to the rear end of the upper body (8) for performing the air input, output operation through a part that is as far away as possible from the aspiration medium.

The aforementioned support ring (6) is designed in rigid structure so as to prevent the aspirator tip (7) coming out of suction end (9) part of the sponge sheath. Thus, the aspirator tip (7) escaping out of the structure during the operation through the end part of the sponge sheath is prevented.

An exemplary embodiment of the aforementioned sponge sheath according to the present invention is presented below with the dimensions thereof. The dimensions are arranged for an exemplary embodiment according to the present invention, wherein said embodiment cannot be used to limit the present invention.

The apparatus is in cylindrical form with a diameter of preferably 10 mm, wherein preferably, the last 3 cm portion thereof is slightly thickened so as to have a diameter of 13 mm in the thickest portion thereof and the shape thereof is rounded so as to form a sponge sheath tip (3), main aspiration channel (5) disposed centrally with a diameter of 4 mm is provided in suction end (9) thereof and said 4 mm diameter main aspiration channel continues with a tunnel having a diameter of 5 mm. The inner hole of the main aspiration channel is fixed to this section and ends with a support ring (6) having a rigid structure preventing the conventional aspirator tip (7) to come out of the sponge having an inner diameter of 4 mm and an outer diameter of 5 mm by being bent. The aspirator channel (2), a tunnel with a diameter of 5 mm, having a cylindrical form, extending from this section through the center of the apparatus until the rear end thereof and in which conventional aspirator tip (7) will be placed is provided. A main aspiration channel (5) with a semi-cylindrical, extending along the edge of the aspirator channel (2) and opening from end to end to the tunnel is provided. The channel is semi-cylindrical in shape and has a diameter of 2 mm. In other words, said channel opens to the aspirator channel (2), in which the aspirator will be placed, along a line having a width of 2 mm and it is a semi-cylindrical channel with a depth of 1 mm. Air channel connection outlet (4) penetrating into the side wall having a diameter of 2 mm is formed on said channel at the point having a distance of 15 mm to the rear end of the apparatus and said hole connects the air channel (1) and the aspirator channel (2) to the exterior of the apparatus. Total length of the apparatus is 13 cm.

The present invention is attached to a conventional aspirator tip (7). During the aspiration, no negative pressure is formed around the conventional aspirator tip (7) provided in the widened end portion of sponge sheath, because, when required, the carbon dioxide gas is replaced through the air channel and by means of the porous structure of the sponge with the fluids removed from this region, thus, omentum and intestinal loops provided inside the abdomen does not block the aspiration by adhering around the aspirator. Consequently, carbon dioxide gas insufflated into the abdomen during laparoscopy through the air channel connection outlet (4) and the air channel (1) reaches the tip of the protective sponge sheath by replacing the fluids being aspirated and formation of the strong negative pressure therein is prevented even if the sponge tip is present deep in the abdomen. Thus, the sponge sheath does not adhere to anything and the aspiration is not blocked. The fluids desired to be aspirated from the interior of the abdomen flow freely into the thickened end portion of the sponge sheath and then, said fluids are removed out of the abdomen by means of the negative pressure formed inside the conventional aspirator.

Preferably, the present invention can also be employed to perform blunt dissection in the tissues. The necessity of having a further instrument for blunt dissection is eliminated and the tool can also be used for exclusions.

1. A sponge sheath for laparoscopic aspirators having a cylindirical upper body (8) characterized by, a sponge sheath tip (3) provided in one end of said upper body (8) and designed in the form of an ellipse having open ends; a main aspiration channel (5) provided in suction end (9) of the sponge sheath tip (3) and designed as a cylindrical channel having a diameter smaller than the diameter of the aspirator channel (2); a support ring (6) positioned between the aspirator channel (2) and the main aspiration channel (5); an air channel (1) positioned in the inner part of the aspirator channel in the form of a smooth semi-cylindrical channel; and an air channel connection outlet (4) connecting the air channel (1) and the aspirator channel (2) to the external environment.

2. Air channel connection outlet (4) according to claim 1, characterized in that it can connect the aspirator channel (2) as well as the air channel (1) to the environment being insufflated with carbon dioxide inside the abdomen.

3. Air channel connection outlet (4) according to claim 1, characterized in that it is positioned in a location being closed to the rear end of the upper body (8).

4. Support ring (6) according to claim 1, characterized in that it is designed in rigid structure so as to prevent the aspirator tip (7) coming out of the end part of the sponge sheath.
